(19) United States
(12) Patent Application Publication
(10) Pub. No.: US 2006/0217788 A1
(43) Pub. Date: Sep. 28, 2006

(54) Method of using laser induced injury to activate topical prodrugs

(76) Inventors: G. Scott Herron, La Honda, CA (US); D. Bommi Bommannan, Los Altos, CA (US)

Correspondence Address: FENWICK & WEST LLP, SILICON VALLEY CENTER, 801 CALIFORNIA STREET, MOUNTAIN VIEW, CA, 94041, US

(21) Appl. No.: 11/318,372

(22) Filed: Dec. 22, 2005

Related U.S. Application Data
(63) Continuation-in-part of application No. 10/888,356, filed on Jul. 9, 2004.
(60) Provisional application No. 60/638,933, filed on Dec. 23, 2004.

(51) Int. Cl. A61N 5/06

(52) U.S. Cl. 607/89

(57) ABSTRACT

Methods and compositions are disclosed for improving the wound healing process and increasing collagen synthesis following laser induced thermal injury. The method comprises delivering prodrugs to a target site before the target site is injured and taking advantage of those enzymes that are physiologically-expressed to promote wound healing to convert the prodrugs to active drugs. The claimed invention has the advantage of the prodrugs being readily available to immediately participate in the wound healing process, but not have any negative side-effects of the active drug being present before injury occurs.

### CROSS-REFERENCE To RELATED APPLICATION

This application claims priority from U.S. Patent Application No. 60/638,933, filed Dec. 23, 2004, and is a continuation-in-part of and claims priority from U.S. application Ser. No. 10/888,356, filed Jul. 9, 2004, which applications are incorporated herein by reference in their entirety.

### FIELD OF THE INVENTION

The invention relates generally to the field of treating medical problems of the skin, and more particularly to the use of prodrugs to treat thermal injury to skin due to laser treatments for skin resurfacing or skin rejuvenation.

### BACKGROUND OF THE INVENTION

Resurfacing and rejuvenation of skin using lasers are some of the most commonly performed procedures in cosmetic and aesthetic surgery. While these procedures do not result in open cuts or significant bleeding, and hence are generally not thought of as wounds, laser procedures do result in significant thermal injury to the skin. In fact, a CO₂ laser skin resurfacing procedure is analogous to a first, and some times a second degree burn where the epidermis is removed by the laser treatment and the dermis is also affected by the thermal injury to the epidermis.

Similar to a mechanical wound, a thermal wound also generally goes through the three healing phases—inflammation, tissue formation (cell proliferation) and tissue remodeling (dermal maturation). Smoller et al. Keratinocyte Protein Expression in Rapidly Regenerating Epidermis Following Laser-induced Thermal Injury, Lasers Surg Med. 9(3):264-70 (1989). As has now been well established, the wound healing process is a highly dynamic process involving mediators, extracellular matrices, blood cells, etc. For a review of the wound healing process, see Singer and Clark, Cutaneous Wound Healing, New England Journal of Medicine 341(10), 738-746 (1999).

Briefly, upon tissue injury, inflammatory leukocytes are recruited to initiate the repair process at the site of injury. Then, monocytes infiltrate the wound site and become activated macrophages that release growth factor and vascular endothelial growth factors. These factors then initiate the formation of granulation tissue. Adherence to the extracellular matrix also stimulates monocytes to differentiate into inflammatory or reparative macrophages. The monocyte- and macrophage-derived growth factors, such as transforming growth factor α, interleukin-1, transforming growth factor β, and insulin-like growth factor I, are typically necessary for the initiation and propagation of new tissue formation in wounds, because macrophage-depleted animals have defective wound repair. Thus, macrophages appear to have a pivotal role in the transition between inflammation and repair.

Reepithelialization of wounds begins within hours after injury. The epidermal cells have to migrate for reepithelialization to occur. The hemidesmosomal links between the epidermis and the basement membrane are dissolved, which allows epidermal cell migration. The degradation of the extracellular matrix, which is required if the epidermal cells are to migrate between the collagenous dermis and the fibrin eschar, depends on the production of collagenase by epidermal cells, as well as the activation of plasmin by plasminogen activator produced by the epidermal cells. Plasminogen activator also activates collagenase (matrix metalloproteinase 1) and therefore facilitates the degradation of collagen and extracellular-matrix proteins. One to two days after injury, epidermal cells at the wound margin begin to proliferate behind the actively migrating cells. Local release of growth factors and increased expression of growth-factor receptors facilitate the migration and proliferation processes. Leading contenders include epidermal growth factor, transforming growth factor α, and keratinocyte growth factor. It will be beneficial to provide these growth factors to the wound site to enhance the repair process.

As the next sequence in the healing process, new stroma, often called granulation tissue, begins to invade the wound space approximately four days after injury. Numerous new capillaries endow the new stroma with its granular appearance. Macrophages, fibroblasts, and endothelial cells move into the wound space at the same time. The macrophages provide a continuing source of growth factors necessary to stimulate fibroplasia and angiogenesis; the fibroblasts produce the new extracellular matrix necessary to support cell ingrowth; and endothelial cells form blood vessels that carry oxygen and nutrients necessary to sustain cell metabolism. Growth factors, especially platelet-derived growth factor and transforming growth factor β1, in concert with the extracellular-matrix molecules, presumably stimulate fibroblasts of the tissue around the wound to proliferate, express appropriate integrin receptors, and migrate into the wound space. It will also be beneficial to provide these growth factors to accelerate the wound healing process.

The structural molecules of newly formed extracellular matrix, termed the provisional matrix, contribute to the formation of granulation tissue by providing a scaffold or conduit for cell migration. These molecules include fibrin, fibronectin, and hyaluronic acid. In fact, the appearance of fibronectin and the appropriate integrin receptors that bind fibronectin, fibrin, or both on fibroblasts appears to be the rate-limiting step in the formation of granulation tissue. The fibroblasts are responsible for the synthesis, deposition, and remodeling of the extracellular matrix. For the extracellular matrix to have a positive effect on the ability of fibroblasts to synthesize, deposit, remodel, and generally interact with the extracellular matrix, it is important that fibronectin and fibrin have to be present in appropriate amounts to promote fibroblast activity. Hence, it will be beneficial to provide fibronectin and fibrin at the wound site to promote wound healing.

Cell movement into a blood clot of cross-linked fibrin or into tightly woven or thermally denatured extracellular matrix may require an active proteolytic system that can cleave a path for cell migration. A variety of fibroblast-derived enzymes, in addition to serum-derived plasmin, are potential candidates for this task, including plasminogen activator, collagenases, gelatinase A, and stromelysin.

After migrating into wounds, fibroblasts commence the synthesis of extracellular matrix. The provisional extracellular matrix is gradually replaced with a collagenous matrix, perhaps as a result of the action of transforming growth factor β1. Once an abundant collagen matrix has been deposited in the wound, the fibroblasts stop producing collagen, and the fibroblast-rich granulation tissue is replaced by a relatively acellular scar.

The formation of new blood vessels is necessary to sustain the newly formed granulation tissue. Angiogenesis is a complex process that relies on extracellular matrix in the wound bed as well as migration and mitogenic stimulation of endothelial cells. The induction of angiogenesis was initially attributed to acidic or basic fibroblast growth factor. Subsequently, many other molecules have also been found to have angiogenic activity, including vascular endothelial growth factor, transforming growth factor β, angiogenin, angiotropin, angiopoietin 1, and thrombospondin, to name but a few. Low oxygen tension and elevated lactic acid may also stimulate angiogenesis. Many of the molecules mentioned above appear to induce angiogenesis by stimulating the production of basic fibroblast growth factor and vascular endothelial growth factor by macrophages and endothelial cells. Activated epidermal cells of the wound secrete large quantities of vascular endothelial-cell growth factor. Basic fibroblast growth factor may set the stage for angiogenesis during the first three days of wound repair, whereas vascular endothelial-cell growth factor is critical for angiogenesis during the formation of granulation tissue on days 4 through 7. Hence, it might be beneficial to provide to the wound site the molecules mentioned above that are capable of stimulating angiogenesis.

The series of events leading to angiogenesis may be as follows. Injury causes destruction of tissue and hypoxia. Angiogenesis factors such as acidic and basic fibroblast growth factor are immediately released from macrophages after cell disruption, and the production of vascular endothelial-cell growth factor by epidermal cells is stimulated by hypoxia. Proteolytic enzymes released into the connective tissue degrade extracellular-matrix proteins. Fragments of these proteins recruit peripheral-blood monocytes to the site of injury, where they become activated macrophages and release angiogenesis factors. Certain macrophage angiogenesis factors, such as basic fibroblast growth factor, stimulate endothelial cells to release plasminogen activator and procollagenase. Plasminogen activator converts plasminogen to plasmin and procollagenase to active collagenase, and in concert these two proteases digest basement membranes. The fragmentation of the basement membrane allows endothelial cells stimulated by angiogenesis factors to migrate and form new blood vessels at the injured site. Once the wound is filled with new granulation tissue, angiogenesis ceases and many of the new blood vessels disintegrate as a result of apoptosis. This programmed cell death probably is regulated by a variety of matrix molecules, such as thrombospondins 1 and 2, and antiangiogenesis factors, such as angiostatin, endostatin, and angiopoietin 2.

What has been described above is the natural biological processes that have been identified to orchestrate the wound healing process, whether they are mechanical or thermal wounds. As noted above, many proteases are involved in the wound healing process. It will be beneficial to take advantage of the synthesis and release of these activators to enhance the wound healing process. Use of prodrugs that get converted to an active drug by these activators is one such approach.

A prodrug is an inactive or partially active drug that is metabolically changed in the body into an active drug. Alternatively, a prodrug may be defined as a chemical which is non-toxic and pharmacodynamically inert, but which can be transformed in vivo into a pharmacologically active drug. Most often, an active drug molecule is conjugated with an enzyme substrate to form the prodrug. These prodrugs are then converted into the active drugs in the presence of the appropriate enzyme which cleaves the link between the enzyme substrate and the active drug.

Some of the common uses of prodrugs are for site-specific delivery where a non-toxic prodrug can be site-specifically activated to cytotoxic drugs using prelocalized or locally available prodrug cleaving catalysts such as enzymes, catalytic antibodies, antibody enzyme conjugates or fusion proteins.

Prodrugs also have the advantages of increased stability, adjusted solubility, improved route of administration, more favorable distribution, improved pharmacokinetics, by-passing resistance, etc. Most prodrugs are administered to the tissue where the cleaving catalysts are already present.

It would be highly desirable to deliver to a target tissue desired prodrugs before the cleaving catalysts are present such that upon the cleaving catalyst being released due to injury or other pathological process the prodrug is now converted to an active drug. For example, in the case of laser induced thermal injury to the skin, it is highly desirable that active drug moieties are available to undertake or facilitate the repair process immediately following the laser induced thermal injury.

### SUMMARY OF THE INVENTION

The present invention overcomes the limitations of the prior art by providing a method of improving the wound healing process following laser induced thermal injury.

In one aspect, the invention provides compositions and methods to augment the wound healing process by administering a prodrug to a target tissue, wherein the prodrug has a moiety that is cleavable by an activator and where the activator is secreted because of injury to the target tissue, and the activator converts the prodrug into an active drug.

In another aspect, the invention provides a composition comprising a prodrug to treat a target tissue having the formula S-A where S is a substrate and A is a drug and S is cleavable by an enzyme, where the substrate is cleaved from the drug only in the presence of an enzyme that is not present in healthy target tissue but only secreted upon injury to the target tissue.

In another embodiment of this invention, a prodrug is delivered to a target tissue in anticipation of a planned injury, wherein the injury releases certain activators in the target tissue and the activators are capable of converting the prodrug into an active drug.

In a further embodiment of this invention, the inventive compositions comprise prodrugs that are activated upon injury to the tissue and are capable of promoting collagen synthesis and angiogenesis in the target tissue.

In yet another embodiment of the claimed invention, the prodrugs are delivered to the target tissue simultaneously along with the thermal injury causing laser energy.

In one aspect, the invention provides a method of activating a prodrug by administering the prodrug to a target tissue, wherein the prodrug has a moiety that is cleavable by an activator wherein the activator is secreted because of injury to the target tissue; and the activator converts the prodrug into active drug. The activator can be an enzyme such as a protease (including metalloproteinase, serine protease and thiol proteinases), a glycosidase, a kinase, a phosphodiesterase, a phosphorylase, a sulfatase, an esterase, a lipase, an oxygenase, a dismutase, a hydroxylase, a ligase, a synthase, or combinations thereof. The active drug can be tetracycline, doxycycline, halofuginone, Periostat, Trocade, FR255031, doxorubicin, N-acetylcysteine, minocycline, colchicine, or combinations thereof.

In another aspect, the invention provides a prodrug to treat a target tissue, wherein the prodrug comprises the formula S-A wherein S is a substrate cleavable by catalytic agent where the substrate is cleaved only in the presence of the catalytic agent that is not present in healthy target tissue but only secreted upon injury to the target tissue; and A is a drug. The catalytic agent can be an enzyme such as a protease (including metalloproteinase, serine protease and thiol proteinases), a glycosidase, a kinase, a phosphodiesterase, a phosphorylase, a sulfatase, an esterase, a lipase, an oxygenase, a dismutase, a hydroxylase, a ligase, a synthase, or combinations thereof. The active drug can be tetracycline, doxycycline, halofuginone, Periostat, Trocade, FR255031, doxorubicin, N-acetylcysteine, minocycline, colchicine, or combinations thereof.

In yet another aspect, the invention provides a method of improving healing of skin wounds by administering a prodrug to the skin wherein the prodrug comprises the formula S-A, wherein S is cleavable by a catalytic agent and A is a drug, and wherein the active agent is present in wounds. S can be a carboxylate, an ester, and amide, or an aldehyde. A can be tetracycline, doxycycline, halofuginone, Periostat, Trocade, FR255031, doxorubicin, N-acetylcysteine, minocycline, colchicine, or combinations thereof. A can also be a growth factors selected from the group consisting of EGF, bFGF, aFGF, TGF-α, TGF-β, KGF, NGF, PDGF, insulin, insulin-like Growth Factors I and II (IGF-I and IGF-II, respectively), Interferons (IFNs), Interleukins (ILs), KGF (Keratinocyte Growth Factor), Macrophage Colony Stimulating Factor (M-CSF), Platelet-Derived Endothelial Cell Growth Factor (PD-ECGF), Stem Cell Factor (SCF), tumor necrosis factor alpha (TNF-α), or combinations thereof. The catalytic agent can be an enzyme such a protease (including metalloproteinase, serine protease and thiol proteinases), a glycosidase, a kinase, a phosphodiesterase, a phosphorylase, a sulfatase, an esterase, a lipase, an oxygenase, a dismutase, a hydroxylase, a ligase, a synthase, or combinations thereof.

Other aspects of the invention include methods corresponding to the compositions and systems described above will become apparent in view of the following description.

### BRIEF DESCRIPTION OF THE DRAWINGS

The invention has other advantages and features which will be more readily apparent from the following detailed description of the invention and the appended claims, when taken in conjunction with the accompanying drawings, in which:

FIG. 1 shows an embodiment of the invention wherein the controlled and targeted release of a drug at or near the site of wound repair following thermal tissue damage is achieved. The system consists of a Prodrug Delivery phase and a Tissue Damage phase which may or may not occur simultaneously. These phases are typically followed by a phase of Production of Catalytic Agent© that is followed by a phase of Activation of Prodrug and Release of Drug A. Drug A is then positioned to act therapeutically at or near the site of wound repair to facilitate various desired effects.

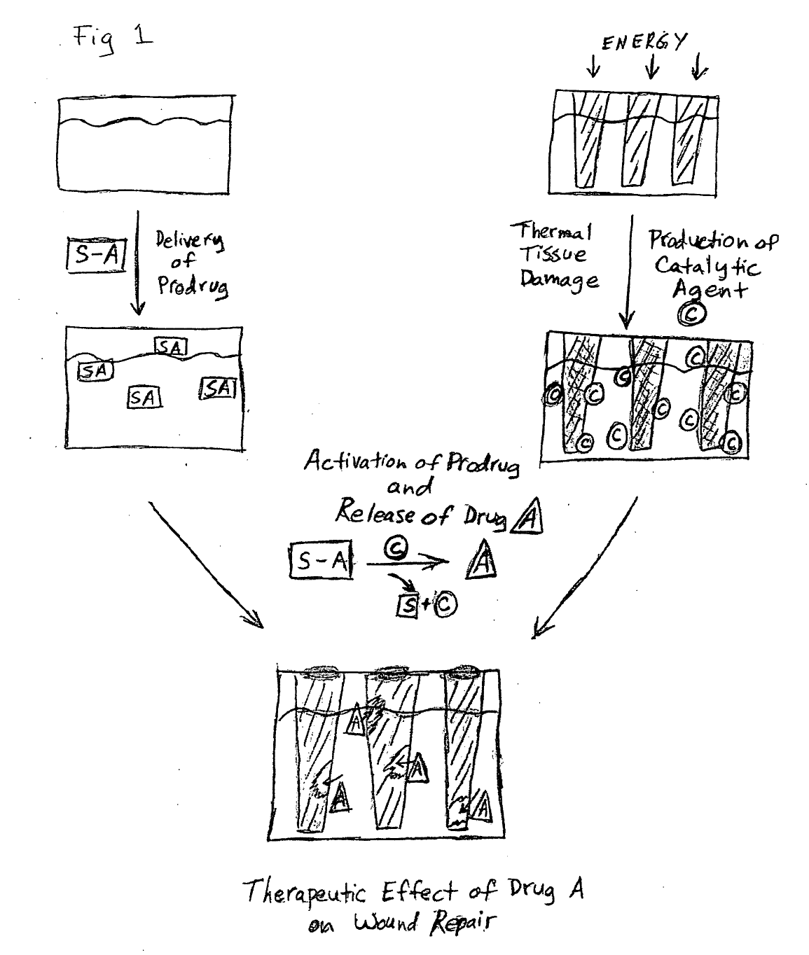

### DETAILED DESCRIPTION

Definitions

Unless otherwise stated, the following terms used in this application, including the specification and claims, have the definitions given below. It must be noted that, as used in the specification and the appended claims, the singular forms “a,” “an” and “the” include plural referents unless the context clearly dictates otherwise. Definition of standard chemistry terms may be found in reference works, including Carey and Sundberg (1992) “Advanced Organic Chemistry 3^{rd} Ed.” Vols. A and B, Plenum Press, New York. The practice of the present invention will employ, unless otherwise indicated, conventional methods of synthetic organic chemistry, mass spectroscopy, preparative and analytical methods of chromatography, protein chemistry, biochemistry, recombinant DNA techniques and pharmacology, within the skill of the art.

The term “wound” as used herein includes but is not limited to thermal injury to the skin, open wound or cuts caused by surgical incision or injury, or burns.

The term “prodrug” as used herein includes a compound that exhibits pharmacological activity after undergoing a chemical transformation in the body, and includes a derivative of the compound which has a chemically or metabolically decomposable group, and shows pharmaceutical activity upon hydrolysis, solvolysis or decomposition under wound healing conditions.

The term “enhancing the reparative phase of wound healing and repair” includes controlling and/or enhancing the chemotaxis of human skin fibroblast tissue cells toward a chemoattractant such as PDGF present at a wound site during the reparative phase of wound healing and/or repair; controlling and/or enhancing collagen synthesis in human skin fibroblast tissue cells which have migrated to and/or are present at a wound site during the reparative phase of wound healing and repair; and inhibiting or dampening the inflammation reaction at a wound site during the reparative phase.

The terms “effective amount” or “pharmaceutically effective amount” refer to a nontoxic but sufficient amount of the agent to provide the desired biological result. That result can be reduction and/or alleviation of the signs, symptoms, or causes of a disease, or any other desired alteration of a biological system. For example, an “effective amount” for therapeutic uses is the amount of the composition comprising a prodrug disclosed herein required to provide a clinically significant increase in wound healing and repair, such as for wounds resulting from a laser skin resurfacing procedure. An appropriate “effective” amount in any individual case may be determined by one of ordinary skill in the art using routine experimentation.

As used herein, the terms “treat” or “treatment” are used interchangeably and are meant to indicate a reduction in the severity of such symptoms that will or are expected to develop, such as those due to laser induced thermal injury. The terms further include ameliorating existing symptoms, preventing additional symptoms, and ameliorating or preventing the underlying metabolic causes of symptoms.

By “pharmaceutically acceptable” or “pharmacologically acceptable” is meant a material which is not biologically or otherwise undesirable, i.e., the material may be administered to an individual without causing any undesirable biological effects or interacting in a deleterious manner with any of the components of the composition in which it is contained.

As used herein, the term “subject” encompasses mammals and non-mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. The term does not denote a particular age or gender.

The present invention relates to methods and compositions for improving wound healing or other beneficial effects in tissue following injury to the tissue, particularly laser induced thermal injury. The method involves administering the desired prodrug to a target tissue before the tissue is injured. The prodrugs have the general formula S-A where S is a substrate that is capable of being cleaved by a catalytic agent© (enzyme involved in wound repair) and A is an active drug that can promote wound healing or other beneficial effects in the tissue. The catalytic agent can be involved in any of the processes of wound healing, such as, for example, (1) inflammation; (2) fibroblast proliferations, collagen synthesis; (3) angiogenesis; (4) wound contracture; and (5) epithelialization.

With few exceptions, MMPs that are expressed in wounded skin are not synthesized in normal skin. Most known MMPs are expressed during wound healing, but their specific biological functions are mainly unknown. MMPs are a multigene family that belongs to the superfamily of metalloproteinases. Altogether 23 human MMPs are known at present, classified as collagenases, gelatinases, stromelysins, membrane-type MMPs, and others. All members of the MMP family are structurally related. Three domains are common to all MMPs: the N-terminal hydrophobic (pre)domain, the propeptide domain, and the catalytic zinc-binding domain. The predomain directs the synthesis of MMPs to the endoplastic reticulum, after which it is removed. The catalytic domain of MMPs has a cleft containing the catalytic Zn²⁺, in which the substrate is bound and then cleaved. MMPs are synthesized as inactive zymogens, with the prodomain masking the catalytic site. A conserved cysteine residue in the prodomain forms a “cysteine switch”, which needs to be disrupted before removal of the propeptide domain and subsequent exposure of catalytic Zn²⁺ are possible. The disengagement of the propeptide may take place through proteolytic processing or as a result of the latent enzyme binding to a ligand or a substrate. Although specific MMPs act on some substrates better than others, many MMPs that are expressed in wounds have overlapping substrate specificities. Substrate selectivity may be directed by the differences in enzyme affinities, and by compartmentalization: activity of a specific MMP is strictly regulated both temporally and spatially during wound repair process.

A prodrug compound that could be cleaved by proteases belonging to the matrix metalloproteinase family (MMPs) can by synthesized by incorporating the pentapeptide KTTKS, copper tripeptide GHK-Cu, and other peptides with PLGLAARK, or another substrate cleavable by MMPs. PLGLAARK is a fairly broad spectrum substrate cleavable by MMP-1, -2, -3, -7, -9 and -13 and has been used successfully in high throughput screening assay systems for MMP activity detection (Peppard J, Pham Q, Clark A, Farley D, Sakane Y, Graves R, George J and Norey C. Assay Drug Dev Technol 1(3): 425-33(2003)). This peptide and similar synthetic substrates are based on peptidase activity studies using the original fluorescence-quenching MMP peptide (7-methoxycoumarin-4-yl) acetyl-L-prolyl-L-leucyl-glycyl-L-leucyl-L-[N3-(2,4 dinitrophenyl)-L-2,3-diaminopropionyl]-L-alanyl-L-arginine amide or MCA-PLGL-A2pr(DNP)AR (Knight C. G., Willenbrock F., Murphy G. FEBS Lett. 296: 263 (1992)).

The pentapeptide KTTKS is a subfragment of the carboxyl-terminal propeptide of type I collagen I. Based on evidence that collagen degradation fragments might act through positive feedback mechanisms to regulate collagen synthesis, in vitro studies showed KTTKS increased production of several matrix components in fibroblast cultures, including collagen I, III and fibronectin (Katayama K, Armendariz-Borunda J, Raghow R, Kang A H and Seyer J. J. Biol. Chem. 268(14):9941-44 (1993)).

The beneficial effects of the copper tripeptide, GHK-Cu, on various wound healing mechanisms, is based on original studies of its extracellular matrix stimulation properties (Maquart F X, Pickart L, Laurent M, Gillery P, Monboisse J C and Borel J P FEBS Lett, 238(2):343-6 (1988)). ProCyte Corporation has marketed a large number of cosmetic formulas incorporating GHK-Cu. Tripeptides as used herein include one or more His-based tripeptides, one or more GHK-tripeptides and/or mixtures thereof. The tripeptides may be naturally occurring or of synthetic origin.

Preferred tripeptides in accordance with one aspect of the present invention are based on the structure Gly-His-Lys and its analogs and derivatives thereof. These are collectively known herein as GHK-tripeptides. Analogs of the preferred tripeptide useful in accordance with the present invention include those in which one or more of the three amino acids are reorganized or rearranged within the sequence (e.g., Gly-Lys-His) and/or where no more than two amino acids are substituted (e.g., His-Ala-Orn).

Derivatives are also considered to be encompassed by the terms pentapeptide and GHK-tripeptides in accordance with the present invention. Derivatives of pentapeptides and GHK-tripeptides include derivatives of the substituted and rearranged peptides, acyl-derivatives which can be derived from acetic acid, capric acid, lauric acid, myristic acid, octanoic acid, palmitic acid, stearic acid, behenic acid, linoleic acid, linolenic acid, lipoic acid, oleic acid, isostearic acid, elaidoic acid, 2-ethylhexaneic acid, coconut oil fatty acid, tallow fatty acid, hardened tallow fatty acid, palm kernel oil fatty acid, lanolin fatty acid and the like. Preferable examples of the acyl group include an acetyl group, a palmitoyl group, an elaidoyl group, a myristyl group, a biotinyl group and an octanoyl group. The acyl group may be unsubstituted or usubstituted, such as with hydroxyl, SO₃H, SH or S—S.

Particularly preferred embodiments of tripeptides in accordance with the present invention include N-Acyl-Gly-His-Lys and most preferably, N-Palmitoyl-Gly-His-Lys. Preferred commercially available tripeptide and tripeptide derivative containing compositions include Biopeptide-CL, Maxilip®, or Biobustyl® all sold by Sederma. Further, the percutaneous absorption of the pentapeptide can be increased by conjugating the pentapeptide to a 16-carbon fatty acid moiety to give Palmitoyl-KTTKS (“Matrixyl” from Sederma SA) or Pal-KTTKS-3 commercially available from Proctor and Gamble in its cosmeceutical line “Regenerist.” Several clinical studies have shown efficacy of Pal-KTTKS at 3 ppm in wrinkle improvement and reduction of photo-aging scores comparable to retinoids but without significant irritation or changes in barrier function (Robinson, L R, Fitzgerald D G, Doughty N C, Dawes N C, Berge C A. and Bissett D L. Int. J. Cos. Sci. 27(3):155-160 (2005)).

A prodrug compound incorporating KTTKS or GHK-Cu can be synthesized by conjugating them to another peptide which is subsequently cleaved by proteases belonging to the matrix metalloproteinase family (MMPs), which are abundant during the inflammatory and remodeling phases of wound repair. Since the peptide PLGLAARK is a fairly broad spectrum substrate cleavable by MMP-1, -2, -3, -7, -9 and -13 (Peppard J, Pham Q, Clark A, Farley D, Sakane Y, Graves R, George J and Norey C. Assay Drug Dev Technol. June;1(3):425-33 (2003)), it could be used to provide a cleavable target for a potential prodrug conjugate.

Thus, in one aspect, the prodrug comprises the sequence PLGLAAR-KTTKS (or PLGLAAR-GHK-Cu), where the dash indicates peptide bonding between the MMP substrate and the cosmeceutical active ingredient. Subsequent cleavage by MMPs can release two fragments, PLG+LAARKTTKS (or LAARGHK-Cu), where the latter peptide can have significant matrix-stimulating and/or wound repair regulatory activity once it interacts with the cellular receptor complexes. Thus, a large number of peptide derivatives can be synthesized as MMP-cleavable prodrug candidates (e.g. PLG˜L-Active Compound) and these can be compared for their relative matrix-stimulating activities. Based on this principle, extended or truncated peptides with various side chains to enhance percutaneous absorption and/or intercellular permeation can be designed to interact with specific receptor complexes to modulate extracellular matrix synthesis during wound repair.

The cosmeceutical active ingredient can include additional peptides, including but not limited to, di-, tri-, tetra-, penta- and hexapeptides and derivatives thereof. Suitable dipeptides for use herein include Carnosine (beta-Ala-His). Suitable tripeptides for use herein include Arg-Lys-Arg, His-Gly-Gly. Preferred tripeptides and derivatives thereof include N-Palmitoyl-Gly-Lys-His, which may be purchased from Sederma; PEPTIDE CK (Arg-Lys-Arg); PEPTIDE CK+(ac-Arg-Lys-Arg-NH₂); and a copper derivative of His-Gly-Gly sold commercially as LAMIN, from Sigma (St. Louis, Mo.). Suitable tetrapeptides for use herein include PEPTIDE E, Arg-Ser-Arg-Lys. Other suitable peptides for use herein include, but are not limited to Tyr-Arg, Val-Trp, Asn-Phe, Asp-Phe, N-Palmitoyl-beta-Ala-His, N-Acetyl-Tyr-Arg-hexadecylester, and derivatives thereof, Lys-Phe-Lys, N-Elaidoyl-Lys-Phe-Lys and its analogs of conservative substitution, N-Acetyl-Arg-Lys-Arg-NH₂, and derivatives thereof. Suitable pentapeptides and hexapeptides for use herein include, but are not limited to N-Palmitoyl-Lys-Thr-Thr-Lys-Ser, N-Palmitoyl-Tyr-Gly-Gly-Phe-X with X Met or Leu or mixtures thereof, N-Palmitoyl-Val-Gly-Val-Ala-Pro-Gly and derivatives thereof. A preferred dipeptide derivative is N-Acetyl-Tyr-Arg-hexadecylester (CALMOSENSINE® from Sederma). Preferred tripeptides and derivatives thereof include N-Palmitoyl-Gly-Lys-His (Pal-GKH from Sederma), Peptide CK (Arg-Lys-Arg) and Lipospondin (N-Elaidoyl-Lys-Phe-Lys) and its conservative substitution analogs, Peptide CK+(N-Acetyl-Arg-Lys-Arg-NH₂). Suitable pentapeptides for use herein also include N-Palmitoyl-Lys-Thr-Thr-Lys-Ser, available as MATRIXYL® from Sederma. Hexapeptides such as those disclosed in French Patent Application No. FR 0305707, filed May 12, 2003, in the name of Sederma may also be used.

Collagen production is vital for the wound healing process. Collagen is the most prevalent protein in animals. It is an obligatory constituent of connective tissues and extra cellular matrices. Collagen networks in the tissues are responsible for establishing and maintaining the physical integrity of diverse extra cellular structures. Collagen, at molecular level, is defined as a protein comprised of lengthy domains of triple-helical confirmation. Collagenous scaffolding of extra cellular matrix includes genetically distinct types of collagen. During the normal wound repair, collagen neosynthesis and deposition of type III collagen is demonstrated in the earliest phase, i.e. 24 hr to 48 hr, period. From that point, a significant increase in type I collagen is associated with the mature wound fibroblasts and subsequent healing events. Because of its important role in the wound healing process, collagen production is a measure of the rate and quality of wound healing. As such, assays that measure collagen production are useful in experimental models to study wound healing.

Together with MMP-8 (collagenase 2) and MMP-13 (collagenase 3), MMP-1 (collagenase-1) participates in the degradation of fibrillar collagens, especially type I collagen, the most abundant protein in the dermis. The importance of MMP-1 in the context of human skin wound healing is well characterized. MMP-I is expressed by the wound edge keratinocytes, in both acute and chronic wounds, and its expression is rapidly shut off after the re-epithelialization is complete. As the wound edge keratinocytes move off the basement membrane, they come to contact with type I collagen in the dermal matrix. The interaction of 21 integrin with type I collagen leads to induction of MMP-1 expression in the wound edge keratinocytes. Furthermore, MMP-1 activity is essential for the onset of keratinocyte migration at the initiation of re-epithelialization.

Collagenase acts by supplementing the natural process for removal of unwanted tissues at the wound site. Collagenase catalyzes the breakdown of collagen and gelatin, and can be used for the treatment of dermal ulcers, including bed sores, venous ulcers, arterial ulcers, and diabetic foot ulcers, as well as for the treatment of severely burned areas, and wound healing.

For example, collagenase (MMP-1) is one such catalytic agent© and its substrate is one substrate and many such substrates are available from different peptide synthesizers. For example, Peptides International in Louisville, Ky. is one such peptide manufacturer.

The gelatinases include two distinct, but highly related, enzymes: a 72-kD enzyme (gelatinase A, HFG, MMP-2) secreted by fibroblasts and a wide variety of other cell types, and a 92-kD enzyme (gelatinase B, HNG, MMP-9) released by mononuclear phagocytes, neutrophils, corneal epithelial cells, tumor cells, cytotrophoblasts and keratinocytes. These gelatinases have been shown to degrade gelatins (denatured collagens), collagen types IV (basement membrane) and V, fibronectin and insoluble elastin.

In addition, aldose reductase inhibitors (ARIS) can be used as wound healing modulators according to the present invention. For example, ARIs, such as those disclosed in U.S. Pat. Nos. 4,717,725, 4,600,717, 4,436,745, and 4,438,272 can be used to improve wound healing. These compounds inhibit the enzyme aldose reductase. The enzyme's inhibition is related to the mechanism of wound healing. ARIs can be used at concentrations between about 0.1 wt. % and 2.0 wt. %.

The active drug A that can inhibit any of these enzymes can be converted into a prodrug using carboxylate groups, ester groups, amide groups, hydroxymethyl groups and aldehyde groups, and their derivatives. Further, active drug A having a N atom can be converted into a prodrug using N-oxide and N-alkyl derivatives.

The active drug A can be one chosen from the list comprising alpha-hydroxy acids, retinol, retinoids, hydroxyacids, growth factors, extracellular matrix components, vitamins, etc., where these molecules are known to promote epidermal turnover, basement membrane remodeling, papillary and reticular dermal repair processes and/or cellular anti-aging. Thus, the active drug A can be, for example, tetracycline, doxycycline, halofuginone, periostat, trocade, FR255031, doxorubicin, N-acetylcysteine, or minocycline, and the like. In addition, the active drug can be colchicine that is known to reduce the synthesis of pro-collagen. Further, the drug can be dimethylaminoethanol (DMAE), StriVectin, and the like.

Preferred growth factors include TGF-β, epidermal growth factor (EGF), fibroblast growth factor (FGF) and platelet derived growth factor (PDGF). Exemplary growth factors include EGF, bFGF, aFGF, TGF-α, TGF-β, KGF, NGF, PDGF, insulin, insulin-like Growth Factors I and II (IGF-I and IGF-II, respectively), Interferons (IFNs), Interleukins (ILs), KGF (Keratinocyte Growth Factor), Macrophage Colony Stimulating Factor (M-CSF), Platelet-Derived Endothelial Cell Growth Factor (PD-ECGF), and Stem Cell Factor (SCF), and tumor necrosis factor alpha (TNF-α) may promote the activation, proliferation, and/or stimulation of cell types involved in the wound healing process. Such growth factors can be used in accordance with the foregoing discussion of this class of wound healing modulators. In addition, the immunomodulators, antiallergics and basement membrane components can be used in combination with these wound healing modulators.

EGF is a polypeptide growth factor (the mature, processed form is 53 amino acids in length [Gray et al., (1983) Nature, vol. 303: 722-25]). In humans, this protein inhibits gastric acid secretion while murine EGF is known to be mitogenic for a number of cell types, including endothelial, epithelial, and fibroblastic cells.

FGF comprises a family of single chain proteins 14-18 kD in size which tightly bind the potent anticoagulant heparin. Two FGF types, acidic and basic, have been reported. The 146 amino acid basic form (bFGF) is more stable and ten times more potent in stimulating mesodermal cells, such as fibroblasts, endothelial cells, and keratinocytes, than acidic FGF (aFGF).

Insulin is a protein hormone secreted by the cells of the pancreatic islets. It is secreted in response to elevated blood levels of glucose, amino acids, fatty acids, and ketone bodies, promoting their efficient storage and use as cellular fuel by modulating the transport of metabolites and ions across cell membranes and by regulating various intracellular biosynthetic pathways. Insulin promotes the entry of glucose, fatty acids, and amino acids into cells. Additionally, it promotes glycogen, protein, and lipid synthesis while inhibiting glucogenesis, glycogen degradation, protein catabolism, and lipolysis. Insulin consists of α and β subunits linked by two disulfide bridges.

IGF-I an IGF-II are members of a growth hormone-dependent family which mediate the effects of growth hormones. These proteins are known to be important in the regulation of skeletal growth. Both molecules have close structural homology to insulin and possess similar biological activities. IGF-I shares a 43% amino acid sequence homology with proinsulin, while IGF-II shares 60% homology with IGF-I. There is essentially no detectable free IGF species present in the blood plasma of mammals. Instead, the IGFs are bound to specific carrier plasma proteins of higher molecular weight. Both IGF species stimulate DNA, RNA, and protein synthesis and are involved in the proliferation, differentiation, and chemotaxis of some cell types. Local administration of IGF-I is known to stimulate the regeneration of peripheral nerves. In addition, IGF-I and PDGF, when administered topically to wounds in pigs, synergize to promote more effective healing than when either factor is administered alone.

Interferons were first identified as proteins that render cells resistant to infection from a wide range of viruses. Three Interferon types have been identified which are produced by activated T and NK (natural killer) cells. A synthetic consensus α-IFN, designed to incorporate regions of commonality among all known α-IFN subtypes, is disclosed in U.S. Pat. No. 4,897,471. All IFNs are growth inhibitory molecules playing an important role in the lymphokine cascade.

The Interleukins (ILs) are a polypeptide family playing a major role in the body's immune response. They are produced by many cell types, particularly T cells, in response to antigenic or mitogenic stimulation. IL-1 is produced following foreign antigen recognition. In addition to mediating the immune response IL-1 is involved in the inflammatory response to acute infection. IL-1 activates B cells and T cells. It induces IL-2 synthesis. It serves as a cofactor in B cell proliferation and differentiation. It enhances T cell and NK cell toxicity. IL-1 also enhances the response of bone marrow progenitors to various colony stimulating factors (CSFs). In inflammation, IL-1 causes bone marrow granulocyte release, serves as a polymononuclear cell chemoattractant, stimulates fibroblast proliferation, and plays a role in collagenase release.

Compounds that are known to have anti-inflammatory properties are also appropriate candidates for active drug A. Antioxidant nutrients, such as vitamin E (mixtures of tocophenols and tocotrienols), flavinoids, L-ascorbic acid and its biologically stable therapeutic derivatives and Coenzyme Q10 are examples of molecules that could promote the cellular expression of the growth factors mentioned above and/or facilitate cellular anti-aging processes.

Methods of synthesizing these prodrugs are known in the art. For example, U.S. Pat. No. 6,681,041 discloses nucleotide-based prodrugs comprising a drug component covalently attached via junctional ester bond(s) to one or more nucleotide components. Release and activation of the drug component of a nucleotide-based prodrug arises from hydrolysis of the junctional ester bond joining the nucleotide component to the drug component. The active drug component may be a nucleoside analog, a nucleic acid ligand, or a non-nucleoside drug. U.S. Pat. No. 6,774,121 discloses prodrugs comprising anti-proliferative drugs covalently linked, via bridging group, to a phospholipid moiety such that the active species is preferentially released, preferably by enzymatic cleavage, at the required site of action.

In one embodiment, the prodrugs of this invention are delivered to skin, the target tissue, using conventional delivery techniques. The prodrugs can be formulated in the form of a cream, lotion or a gel and applied to the surface of the skin. Alternatively, they can be formulated into a solution and can be injected at the site where the injury is planned. For example, for laser skin resurfacing the prodrug formulation in a conventional formulation can be applied to the face before laser treatment. The inflammatory phase of the healing process is initiated by the body immediately following the laser thermal injury. The degradation of the denatured collagen and extracellular matrix proteins is initiated by the activation of collagenase (and other matrix metalloproteinases-MMPs). This increase in collagenase concentration can now cleave the bonding between the collagenase substrate and the active drug in the prodrug. If the active drug is a small molecule activator or inhibitor of a signaling pathway that stimulates the expression of other beneficial growth factors in the repair process, then wound healing is enhanced.

The desired topical formulation may also contain skin penetration enhancers that are commonly used to increase the permeability of skin and thereby enhance transdermal permeation. Both naturally occurring and synthetic skin permeation enhancers could be used. Long chain fatty acids, such as oleic acid, ceramides, squalenes, d-limonene, monoglyceride and ethyl palmitate, propylene glycol and many other compounds have been reported to enhance the permeation of molecules across the skin. For a review of enhancers that have been reported to be successful in increasing skin permeation, see reviews by Purdon et al., Penetration enhancement of transdermal delivery current permutations and limitations, Crit Rev Ther Drug Carrier Syst. 2004:21(2):97-132; Sinha and Kaur, Permeation enhancers for transdermal drug delivery, Drug Dev Ind Pharm. 2000 November;26(11):1131-40.

The prodrugs could also be loaded into polymeric microparticles, where such polymers could be biodegradable, such as polylactide or polyanhydride, or non-biodegradable. The prodrugs could also be loaded into liposomal delivery system. The choice of the delivery system will be dictated by how lipophilic or hydrophilic the prodrug is and whether any sustained release profile is desired. As the repair process is temporal, with the inflammatory phase lasting for hours, the tissue formation lasting for days and the tissue remodeling extending to weeks and months, it might be beneficial to take advantage of the controlled delivery capabilities of the drug delivery systems. For example, for those prodrugs that influence the inflammatory phase, a system that enhances permeation may be the most effective. Prodrugs that influence the tissue formation process might benefit from the sustained release biodegradable system that lasts a few days. Prodrugs that influence the tissue remodeling phase might have a significant impact if the prodrug were to slowly elute out of a carrier that degrades over weeks.

The laser injury could be caused any type of conventional laser treatment using CO₂, Er-YAG and other such commonly used lasers. The laser induced thermal injury could also be due to the tissue sparing FRAXEL™ laser type treatment provided by the FRAXEL™ laser system manufactured by Reliant Technologies, Inc., of Palo Alto, Calif. In a FRAXEL™laser treatment, laser microspots are scanned on the skin surface. Because a pre-determined fraction of the skin is not exposed to the laser energy, skin that is not exposed to laser energy is spared from thermal necrosis. This spared tissue then participates in and promotes healing of the tissue. Unlike CO₂ treatment, where the entire layer of the skin, primarily epidermis, is ablated and hence results in a difficult and prolonged regeneration of the skin, the tissue sparing FRAXEL™ treatment promotes faster and more robust healing of the skin. The inventive method claimed here can assist skin rejuvenation after any kind of laser induced thermal injury.

In terms of the method of delivery, the prodrugs are preferably delivered before the occurrence of any skin injury, particularly laser induced thermal injury. One objective of the method is for the prodrugs to be readily available to participate in the repair process immediately following the injury. Because of the nature of the prodrugs, they are inactive until they are converted by some metabolic action into the active drug. Here we are relying upon the expression of the enzymes, such as collagenase, that orchestrate the repair process following injury, whether mechanical or thermal skin injury. The enzymes that are expressed in abundance following injury then convert the prodrugs from their inactive forms by cleaving the active drug that was linked to the enzyme substrate.

Many of the active molecules, drug A, that were mentioned earlier may not be efficacious or have side effects in the absence of injured skin. Others, while efficacious, might have side effects in their native form. Delivering such active molecules in the prodrug form might overcome these issues. As mentioned earlier, the prodrugs are inactive until the appropriate enzyme or mediator becomes available to cleave the substrate from the active drug.

Although the detailed description contains many specifics, these should not be construed as limiting the scope of the invention but merely as illustrating different examples and aspects of the invention. It should be appreciated that the scope of the invention includes other embodiments not discussed in detail above. Various other modifications, changes and variations which will be apparent to those skilled in the art may be made in the arrangement, operation and details of the method and apparatus of the present invention disclosed herein without departing from the spirit and scope of the invention as defined in the appended claims. Therefore, the scope of the invention should be determined by the appended claims and their legal equivalents. Furthermore, no element, component or method step is intended to be dedicated to the public regardless of whether the element, component or method step is explicitly recited in the claims.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

### Example 1

The composition of a representative prodrug containing lotion used for a method of the present invention is shown below.

| Ingredients | % w/w |
|---|---|
| Water | 87.15 |
| glycerin | 1.00 |
| xanthan gum | 0.50 |
| octyl palmitate | 10.00 |
| palmitic acid | 0.25 |
| stearic acid | 0.25% |
| PLGLAARK-GHK:copper(II) | 0.30 |
| propylene glycol | 0.55 |
| Total | 100.00 |

The ingredients are mixed to provide a lotion. The utility of the lotion used for a method of the present invention is demonstrated in an 8 week study involving 10 subjects, where the GHK-Cu containing prodrug is tested for its effect on wound healing. 4 of the subjects are used as a control group that apply the lotion that lacks the prodrug. The lotion is applied as a thin film to the faces of the subjects, and then laser microspots are scanned on the skin surface using the FRAXEL™ laser treatment where a pre-determined fraction of the skin is not exposed to the laser energy. Optionally, the lotion can be applied to skin surface plaques twice each day, once in the morning and once at night, over the duration of the study.

At the beginning of the study, and at weeks 4 and 8, the amount and extent of injury due to laser injury is assessed by a clinician. The effect of the lotion used for the method of the present invention on injury and its effect on the healing process is assessed over the 8 weeks. The results of the evaluation of wound healing shows that all of the subjects respond to treatment with a reduction in the time required to heal. The control group shows greater injury immediately after the laser treatment. Further, the injury in the study group given the lotion containing the prodrug heals to a greater extent at the end of the study compared with the control group. Thus, the above examples demonstrate the effectiveness of the method the present invention in promoting the healing process.

All printed patents and publications referred to in this application are hereby incorporated herein in their entirety by this reference.

While the preferred embodiment of the invention has been illustrated and described, it will be appreciated that various changes can be made therein without departing from the spirit and scope of the invention.

In the claims, reference to an element in the singular is not intended to mean “one and only one” unless explicitly stated, but rather is meant to mean “one or more.” In addition, it is not necessary for a device or method to address every problem that is solvable by different embodiments of the invention in order to be encompassed by the claims.

What is claimed is:

1. A method of activating a prodrug comprising:
administering the prodrug to a target tissue, wherein the prodrug has a moiety that is cleavable by an activator wherein the activator is secreted because of injury to the target tissue; and
the activator converts the prodrug into active drug.

2. The method of claim 1, where the target tissue is skin.

3. The method of claim 2, wherein the injury is due to laser exposure to the skin.

4. The method of claim 3, wherein the activator is an enzyme.

5. The method of claim 4, wherein the enzyme is selected from the group consisting of a protease (including metalloproteinase, serine protease and thiol proteinases), a glycosidase, a kinase, a phosphodiesterase, a phosphorylase, a sulfatase, an esterase, a lipase, an oxygenase, a dismutase, a hydroxylase, a ligase and a synthase, or combinations thereof.

6. A prodrug to treat a target tissue, wherein the prodrug comprises the formula
S-A
wherein S is a substrate cleavable by catalytic agent where the substrate is cleaved only in the presence of the catalytic agent that is not present in healthy target tissue but only secreted upon injury to the target tissue; and
A is a drug.

7. The prodrug of claim 6, where the target tissue is skin.

8. The prodrug of claim 7, where the drug is selected from the list consisting of tetracycline, doxycycline, halofuginone, Periostat, Trocade, FR255031, doxorubicin, N-acetylcysteine, minocycline, and colchicine, or combinations thereof.

9. The prodrug of claim 8, where the injury is laser exposure to the skin.

10. The prodrug of claim 9, wherein the catalytic agent is an enzyme selected from the group consisting of a protease, glycosidase, a kinase, a phosphodiesterase, a phosphorylase, a sulfatase, an esterase, a lipase, an oxygenase, a dismutase, a hydroxylase, a ligase and a synthase, or combinations thereof.

11. The prodrug of claim 10, wherein the enzyme is a protease selected from the group consisting of metalloproteinase, serine protease and thiol proteinase.

12. The prodrug of claim 11, wherein the enzyme is a metalloproteinase.

13. The prodrug of claim 12, wherein the metalloproteinase is collagenase.

14. The prodrug of claim 1, wherein S is PLGLAARK (SEQ ID NO: 2).

15. The prodrug of claim 14, wherein A is a pentapeptide or a tripeptide.

16. The prodrug of claim 15, wherein A is KTTKS (SEQ ID NO: 1), GHK, GHK-Cu, analogs, derivatives or combinations thereof.

17. The prodrug of claim 14, wherein A further comprises an acyl group.

18. The prodrug of claim 17, wherein the acyl group is palmitoyl group, elaidoyl group, or myrityl group.

19. A method of improving healing of skin wounds, the method comprising:
administering a prodrug to the skin wherein the prodrug comprises the formula S-A, wherein S is cleavable by a catalytic agent and A is a drug, and wherein the active agent is present in wounds.

20. The method of claim 19, wherein the S is a carboxylate, an ester, and amide, or an aldehyde.

21. The method of claim 20, wherein S is a carboxylate.

22. The method of claim 19, wherein A is selected from the list consisting of tetracycline, doxycycline, halofuginone, Periostat, Trocade, FR255031, doxorubicin, N-acetylcysteine, minocycline, and colchicine, or combinations thereof.

23. The method of claim 22, wherein A is tetracycline.

24. The method of claim 22, wherein A is doxycycline.

25. The method of claim 22, wherein A is doxorubicin.

26. The method of claim 19, wherein the active agent is an enzyme selected from the group consisting of a protease, glycosidase, a kinase, a phosphodiesterase, a phosphorylase, a sulfatase, an esterase, a lipase, an oxygenase, a dismutase, a hydroxylase, a ligase and a synthase, or combinations thereof.

27. The method of claim 26, wherein the enzyme is a protease selected from the group consisting of metalloproteinase, serine protease and thiol proteinase.

28. The method of claim 27, wherein the enzyme is a metalloproteinase.

29. The method of claim 28, wherein the metalloproteinase is collagenase.

30. The method of claim 19, where the skin wound is due to laser exposure.

31. The method of claim 19, wherein A is a growth factors selected from the group consisting of EGF, bFGF, aFGF, TGF-α, TGF-β, KGF, NGF, PDGF, insulin, insulin-like Growth Factors I and II (IGF-I and IGF-II, respectively), interferons (IFNs), Interleukins (ILs), KGF (Keratinocyte Growth Factor), Macrophage Colony Stimulating Factor (M-CSF), Platelet-Derived Endothelial Cell Growth Factor (PD-ECGF), Stem Cell Factor (SCF), and tumor necrosis factor alpha (TNF-α), or combinations thereof.

32. The method of claim 19, wherein S is PLGLAARK (SEQ ID NO: 2).

33. The method of claim 32, wherein A is a pentapeptide or a tripeptide.

34. The method of claim 33, wherein A is KTTKS (SEQ ID NO: 1), GHK, GHK-Cu, analogs, derivatives or combinations thereof.

35. The method of claim 32, wherein A further comprises an acyl group.

36. The method of claim 35, wherein the acyl group is palmitoyl group, elaidoyl group, or myrityl group.


SEQUENCE LISTING (10 sequences)

SEQ ID NO: 1 - protein, 5 aa, Artificial Sequence
  Description of Artificial Sequence Synthetic peptide
KTTKS

SEQ ID NO: 2 - protein, 8 aa, Artificial Sequence
  Description of Artificial Sequence Synthetic peptide
PLGLAARK

SEQ ID NO: 3 - protein, 12 aa, Artificial Sequence
  Description of Artificial Sequence Synthetic peptide
PLGLAARKTTKS

SEQ ID NO: 4 - protein, 10 aa, Artificial Sequence
  Description of Artificial Sequence Synthetic peptide
PLGLAARGHK

SEQ ID NO: 5 - protein, 9 aa, Artificial Sequence
  Description of Artificial Sequence Synthetic peptide
LAARKTTKS

SEQ ID NO: 6 - protein, 7 aa, Artificial Sequence
  Description of Artificial Sequence Synthetic peptide
LAARGHK

SEQ ID NO: 7 - protein, 4 aa, Artificial Sequence
  Description of Artificial Sequence Synthetic peptide
RSRK

SEQ ID NO: 8 - protein, 5 aa, Artificial Sequence
  Description of Artificial Sequence Synthetic peptide
YGGFX

SEQ ID NO: 9 - protein, 6 aa, Artificial Sequence
  Description of Artificial Sequence Synthetic peptide
VGVAPG

SEQ ID NO: 10 - protein, 11 aa, Artificial Sequence
  Description of Artificial Sequence Synthetic peptide
PLGLAARKGHK